(19) United States
(12) Patent Application Publication
(10) Pub. No.: US 2021/0186748 A1
(43) Pub. Date: Jun. 24, 2021

(54) GERMICIDAL FORCED-AIR MEDICAL WARMING SYSTEM

(71) Applicant: David Louis Kirschman, Dayton, OH (US)

(72) Inventor: David Louis Kirschman, Dayton, OH (US)

(73) Assignee: Aerobiotix, Inc., Miamisburg, OH (US)

(21) Appl. No.: 17/012,694

(22) Filed: Sep. 4, 2020

Related U.S. Application Data
(60) Provisional application No. 62/950,532, filed on Dec. 19, 2019.

(51) Int. Cl. A61F 7/00; A61L 9/20

(52) U.S. Cl. CPC A61F 7/0097; A61F 7/0085; A61L 2209/12; A61F 2007/006; A61L 9/20

(57) ABSTRACT

A medical warming system and warming blanket having decontamination means or apparatus for germicidal decontamination of air passing into and/or through the warming blanket.

### CROSS-REFERENCE TO RELATED APPLICATION

The present application claims priority to provisional U.S. Application Ser. No. 62/950,532, filed Dec. 19, 2019, to which Applicant claims the benefit of the earlier filing date. This provisional application is incorporated herein by reference and made a part hereof.

### BACKGROUND OF THE INVENTION

### 1. Field of the Invention

This invention relates to a warming blanket that has decontamination means or apparatus for treating air that passes into and/or through the warming blanket.

### 2. Description of the Related Art

In medical and surgical settings there is a frequent need to modulate the body temperature of the patient. Commonly, in surgical procedures, patient body temperature can fall due to physiological and anatomical factors. This hypothermia can increase the risk for bleeding, infection and other significant complications. Forced-air patient warming has become the standard for maintaining patient body temperature during surgical procedures. These systems typically consist of an air-handling unit comprising a blower, heating element, controller, connection tube, and a flexible blanket containing air chambers. In a typical embodiment, the forced-air blanket comprises small apertures to allow for warmed air to exit the blanket and create a convective heating current around the patient.

A recent concern in such systems is that the convective air can contain potential microbial contamination which can contribute to the risk of surgical site infections. This contamination can arise due to room air contamination entering the air handler, or convective currents aerosolizing microbial particles from the patients themselves. Some systems have attempted to reduce contamination risk by introducing mechanical filtration into the air handler, and/or blanket, at the point of the convective apertures. However, mechanical air filtration can be subject to leakage, contamination of the filter itself, and transmission of small particles, such as viruses. Filters also serve to increase static backpressure and reduce system efficiency.

What is needed, therefore, is an improved system and method for decontaminating and removing germicides from air delivered to or received from a warming means or apparatus, such as a warming blanket.

### SUMMARY OF THE INVENTION

Air disinfection using irradiation, such as C-Band ultraviolet, when correctly applied, has been shown to significantly reduce microbial levels in a moving air column.

It is one object of this invention to apply principles of airstream irradiation to forced-air patient warming systems, in several embodiments.

Another object of the invention is to provide a warming system that also comprises a germicidal decontamination system or means for providing decontaminated air to and/or through a blanket.

Another object of the invention is to provide a system and method for warming and decontaminating air wherein the germicidal decontamination system is situated in the air handler.

Another object of the invention is to provide a system and method for warming and decontaminating air wherein the germicidal decontamination system is situated either upstream or downstream of the patient and separate from the air handler.

Another object of the invention is to provide a warming and decontaminating system and method that provides decontaminated and heated air to a patient.

Yet another object of the invention is to provide a warming and decontamination system that is adapted to deliver warmed and decontaminated air to a patient and collect the air at or near the patient for purposes of further decontamination.

Another object of the invention is to provide a warming blanket and decontamination system that provides germicidal decontamination so that air exiting the blanket into a surrounding environment is substantially decontaminated.

Still another object of the invention is to provide a decontamination system that is adapted for use with an air handler and/or a warming blanket in order to treat or decontaminate the air prior to it reaching the patient.

Another object of the invention is to provide an ultraviolet germicidal decontamination system that may be situated inside of an air handler or outside of an air handler and is adapted for decontaminating air.

Yet another object of the invention is to provide a warming system having a decontamination or germicidal decontamination system having means for controlling air flow and/or pressure in the germicidal decontamination system.

Another object of the invention is to provide a warming system having a decontamination or germicidal decontamination system having means and apparatus for controlling air flow in the germicidal decontamination system.

Another object of the invention is to provide a warming system having means or apparatus for disrupting air flow in order to maximize air exposure time to at least one or a plurality of decontaminating or germicidal removal means, such as at least one or a plurality of ultraviolet lamps in order to improve decontamination efficiency.

Still another object of the invention is to provide a warming system having a collection system for collecting or scavenging air at or near the patient and collecting air that is exhausted from a warming blanket, and if desired, returning the air to a decontamination system.

In one aspect, one embodiment of the invention comprises a medical warming system comprising an air handler; a heater for heating air generated or received by the air handler; a blanket adapted to be placed on or near a patient, the blanket having at least one air channel for receiving an air stream generated by the air handler, the blanket having at least one perforation, exhaust or aperture for permitting at least some of the air stream received by the blanket to exit the blanket; and a germicidal radiation source for decontaminating the air stream generated or received by the air handler.

In another aspect, another embodiment of the invention comprises a medical warming system comprising an air handler; a heater for heating an air stream generated or received by the air handler; a blanket adapted to be placed on or near a patient, the blanket having at least one air channel for receiving the air stream generated by the air handler, the blanket having at least one exhaust for permitting at least some of the air stream received by the blanket to exit the blanket; and a germicidal radiation source for decontaminating the air stream generated or received by the air handler; the exhaust comprising the germicidal radiation source which irradiates the air stream after the air stream passes through the blanket and prior to the air stream being exhausted from the medical warming system.

In another aspect, another embodiment of the invention comprises a medical warming system comprising an air handler; a heater for heating an air stream generated or received by the air handler; a warming blanket adapted to be placed on or near a patient, the warming blanket having at least one air channel for receiving the air stream generated by the air handler, the warming blanket having at least one perforation, exhaust or aperture for permitting at least some of the air stream received by the warming blanket to exit the warming blanket; a germicidal radiation source for decontaminating the air stream generated or received by the air handler; a communication conduit comprising a delivery conduit for coupling an outlet of the air handler to an inlet of the at least one air channel of the warming blanket and a return conduit for coupling an outlet of the at least one air channel of the warming blanket to an inlet of the air handler, the germicidal radiation source being situated in at least one of the delivery conduit or the return conduit.

In another aspect, another embodiment of the invention comprises a medical warming system comprising an air handler; a warming blanket adapted to be placed on or near a patient, the warming blanket having at least one air channel for receiving an air stream generated by the air handler, the warming blanket having at least one perforation, exhaust or aperture for permitting at least some of the air stream received by the warming blanket to exit the warming blanket; and a germicidal radiation source for decontaminating the air stream generated or received by the air handler; the air handler generating the air stream that is in two way communication between the air handler and the warming blanket, so that the air handler generates an outgoing air stream and substantially simultaneously receives an incoming air stream from the warming blanket; the germicidal radiation source irradiating both the incoming air stream into the air handler and the outgoing air stream from the air handler substantially simultaneously.

In another aspect, another embodiment of the invention comprises a medical warming system comprising an air handler; a warming blanket adapted to be placed on or near a patient, the warming blanket having at least one air channel for receiving an air stream generated by the air handler, the warming blanket having at least one perforation, exhaust or aperture for permitting at least some of the air stream received by the warming blanket to exit by warming blanket via positive pressure; a germicidal radiation source for decontaminating by air stream generated or received by the air handler; and a scavenger or collector in fluid communication with the air handler, the scavenger or collector being situated in proximity to the patient and capturing the air stream under negative pressure generated by the air handler and returning the air stream to the air handler, the scavenger or collector comprising the germicidal radiation source which irradiates the air stream collected by the scavenger or collector.

In another aspect, another embodiment of the invention comprises a medical forced air warming system comprising an air handler comprising a warming means; a warming blanket with air channels; an air stream between and inclusive of the air handler and the blanket; and a germicidal radiation source; the radiation source being in communication with the airstream.

In another aspect, another embodiment of the invention comprises a medical forced air warming system comprising an air handler comprising a warming means; a warming blanket with air channels; an air stream between and inclusive of the air handler and the blanket; and an exhaust means to allow the air stream to exit the blanket; the exhaust means comprising a germicidal radiation source which irradiates the air prior to exhausting from the system.

In another aspect, another embodiment of the invention comprises a medical forced air warming system comprising an air handler comprising a warming means; a warming blanket with air channels; and an air stream in two way communication between the air handler and the blanket; the communication comprising a return to the air handler; the air handler further comprising a germicidal irradiation means to irradiate the air stream retuning from the blanket.

In another aspect, another embodiment of the invention comprises a medical forced air warming system comprising an air handler comprising a warming means; a warming blanket with air channels; and an air stream in two way communication between the air handler and the blanket; the air handler handling outgoing and incoming air in communication with the blanket; the air handler further comprising a germicidal irradiation means irradiating the incoming and the outgoing air in a substantially simultaneous manner.

In another aspect, another embodiment of the invention comprises a medical forced air warming system comprising an air handler comprising a warming means; a warming blanket with air channels; an air stream between and inclusive of the air handler and the blanket; and a germicidal radiation means; the radiation means in communication with the airstream; the radiation means comprising a radiation source in cooperation with air baffles, lenses, blockers, reflective surfaces, or similar means to optimize irradiation dosage and/or exposure duration of the air stream and/or suspended microbes therein.

In another aspect, another embodiment of the invention comprises a medical forced air warming system comprising an air handler comprising a warming means; a warming blanket comprising air channels; a substantially tubular connection between the air handler and the blanket; and a germicidal radiation source; the radiation source mounted within, or in communication with, the tubular connection.

In another aspect, another embodiment of the invention comprises a medical forced air warming system comprising an air handler comprising a warming means; a warming blanket comprising air channels; an air stream between and inclusive of the air handler and the blanket; and an exhaust means to allow the air stream to exit the blanket; the exhaust means comprising a pressure valve and communicating with a germicidal radiation source which irradiates the air prior to exhausting from the system.

In another aspect, another embodiment of the invention comprises a medical forced air warming system comprising an air handler comprising a warming means; a warming blanket comprising air channels; an air stream between and inclusive of the air handler and the blanket; and an exhaust means to allow the air stream to exit the blanket; the exhaust means comprising a pressure valve and communicating with a germicidal radiation source which irradiates the air; the pressure valve being adjustable to allow for level of air return versus release from the blanket.

In another aspect, another embodiment of the invention comprises a medical forced air warming system comprising an air handler comprising a warming means; a warming blanket comprising air apertures under positive air pressure; and a scavenging means comprising air apertures under negative air pressure; the scavenging means being in proximity to a subject being warmed and comprising a germicidal radiation source which irradiates the scavenged air.

This invention, including all embodiments shown and described herein, could be used alone or together and/or in combination with one or more of the features covered by one or more of the following list of features:
The medical warming system wherein the germicidal radiation source is located upstream of the patient so that the air stream generated by the air handler is decontaminated before the air stream reaches the blanket.
The medical warming system wherein the germicidal radiation source is located downstream of the patient so that the air stream is decontaminated by the germicidal radiation source after the air stream passes through the blanket.
The medical warming system wherein the heater and the germicidal radiation source are situated in the air handler.
The medical warming system wherein the medical warming system further comprises a conduit or air tube for receiving the air stream generated by the air handler and for delivering the air stream to the at least one air channel of the blanket, the germicidal radiation source being in fluid communication with the conduit or air tube and situated between the air handler and the blanket and irradiating at least some of the air stream generated by the air handler before it enters in the blanket.
The medical warming system wherein the medical warming system further comprises a conduit or air tube for receiving the air stream generated by the air handler and for delivering the air stream to the at least one air channel of the blanket and, thereafter, to an environment, the germicidal radiation source being in fluid communication with the conduit or air tube and adapted to receive and irradiate at least a portion of the air stream after the air stream exits the blanket and before the air stream is introduced into the environment.
The medical warming system wherein the conduit or air tube comprises at least one adjustable valve for controlling pressure or airflow through the blanket.
The medical warming system wherein the germicidal radiation source comprises an irradiation optimizer for optimizing irradiation dosage and/or exposure duration of the air stream to irradiate microbes suspended therein.
The medical warming system wherein the irradiation optimizer comprises at least one of air baffles, airflow interrupters, lenses, blockers or reflective surfaces.
The medical warming system wherein the germicidal radiation source irradiates the air stream such that substantially decontaminated air is introduced into an environment around the patient.
The medical warming system wherein the germicidal radiation source comprises at least one ultraviolet lamp.
The medical warming system wherein the germicidal radiation source comprises a plurality of ultraviolet lamps.
The medical warming system wherein the germicidal radiation source is situated in the air handler.
The medical warming system wherein the medical warming system further comprises a conduit or air tube for receiving the air stream generated by the air handler and for delivering the air stream to the at least one air channel of the blanket and, thereafter to an environment, the germicidal radiation source being in fluid communication with the conduit or air tube and adapted to receive and irradiate at least a portion of the air stream after the air stream exits the blanket and before the air stream is exhausted from the medical warming system.
The medical warming system wherein the germicidal radiation source is located in the return conduit so that the air stream is decontaminated by the germicidal radiation source after the air stream passes through the warming blanket and before the air stream passes into the inlet of the air handler.
The medical warming system wherein the medical warming system further comprises a conduit or air tube for receiving the air stream generated by the air handler and for delivering the air stream to the at least one air channel of the warming blanket and, thereafter to an environment, the germicidal radiation source being in fluid communication with the conduit or air tube and adapted to receive and irradiate at least a portion of the air stream after the air stream exits the warming blanket and before the air stream is exhausted from the medical warming system.
The medical warming system wherein the medical warming system comprises at least one adjustable valve for controlling pressure or airflow through the warming blanket.
The medical warming system wherein the at least one adjustable valve is situated in the return conduit.
The medical warming system wherein the medical warming system comprises a communication conduit comprising a delivery conduit for coupling an outlet of the air handler to an inlet of the at least one air channel of the warming blanket and a return conduit for coupling an outlet of the at least one channel of the warming blanket to an inlet of the air handler, the germicidal radiation source being situated in at least one of the delivery conduit or the return conduit.
The medical warming system wherein the germicidal radiation source is located in the air handler so both air entering and exiting the air handler are subject to irradiation by the germicidal radiation source.
The medical warming system wherein the medical warming system further comprises a heater situated in the air handler for heating the air stream generated by the air handler.
The medical warming system wherein the germicidal radiation source is located in the air handler and irradiates the air stream received in the air handler so that the air stream is decontaminated by the germicidal radiation source after the air stream passes through the warming blanket and before the air stream passes out of the air handler.
The medical warming system wherein the medical warming system comprises a collector conduit coupling the scavenger or collector to the air handler, the germicidal radiation source being in communication with the collector conduit so that the air stream is decontaminated by the germicidal radiation source after the air stream passes through the warming blanket and before the air stream passes into the air handler.
The medical warming system wherein the scavenger or collector comprises a plurality of inlets for capturing the air stream and for delivering the air stream to the germicidal radiation source where the air stream is irradiated.
The medical warming system wherein a system heater is located in the air handler for heating the air stream.
The medical warming system wherein a system heater is located in the warming blanket.
The medical warming system wherein the medical warming system comprises a communication conduit comprising a delivery conduit for coupling an outlet of the air handler to an inlet of the at least one air channel of the warming blanket and a return conduit for coupling to an outlet of the scavenger or collector to an inlet of at least one of an inlet of the air handler or an inlet of the germicidal radiation source.
The medical warming system wherein the scavenger or collector comprises scavenging means for scavenging and collecting air discharged from the warming blanket, the scavenging means comprising the germicidal radiation source and a perforated generally planar collector.
The medical warming system wherein the germicidal radiation source is located in the collector conduit so that the air stream is decontaminated by the germicidal radiation source after the air stream passes through the warming blanket and before the air stream passes into an inlet of the air handler.
The medical warming system wherein the medical warming system further comprises a conduit or air tube for receiving the air stream generated by the air handler and for delivering the air stream to the at least one air channel of the warming blanket and, thereafter, to the germicidal radiation source which is in fluid communication with the conduit or air tube and adapted to receive and irradiate at least a portion of the air stream after the air stream exits the warming blanket.

These and other objects and advantages of the invention will be apparent from the following description, the accompanying drawings and the appended claims.

### BRIEF DESCRIPTION OF THE ACCOMPANYING DRAWINGS

FIG. 1 is a schematic illustration of a prior art warming blanket;

FIG. 2 illustrates an embodiment of the invention showing a warming blanket with germicidal decontamination means and system that are situated in an air handler;

FIG. 3 illustrates a further embodiment of the invention showing a warming blanket with germicidal decontamination means, apparatus and system that are situated outside of the air handler and upstream of the patient;

FIG. 4 illustrates a further embodiment of the invention showing a warming blanket with germicidal decontamination means, apparatus and system that are situated outside of the air handler and downstream of the patient;

FIG. 5 is a view of another embodiment illustrating a configuration wherein at least some of the air exhausted from the warming blanket which is controlled by a pressure valve returns to the air handler and the germicidal decontamination means, apparatus and system which irradiates the input and output lines and also has a pressure valve that controls conductive versus convective airflow;

FIG. 6 is a view of another embodiment illustrating a configuration wherein the exhausted air from the warming blanket is scavenged or captured and returned to the air handler via a separate scavenger or collector with irradiation and recycling of the air after it leaves the warming blanket; and

FIG. 7 is a view of another embodiment illustrating a configuration wherein the exhausted air from the warming blanket is returned to the air handler, which creates both positive and negative pressure in the system, via a separate scavenger or collector and there is simultaneous irradiation of both the return and exit lines.

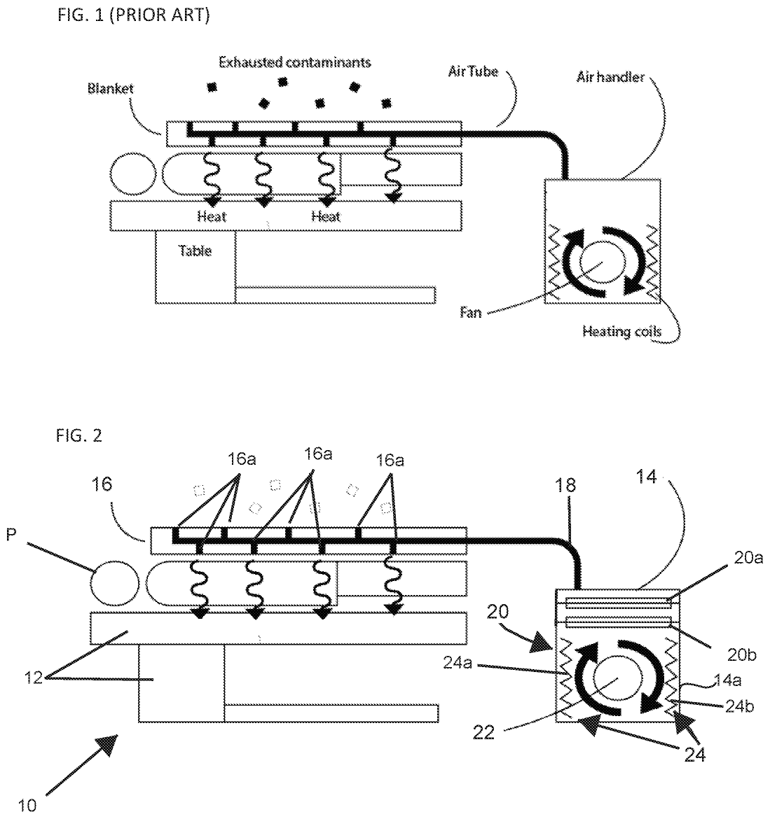

### DETAILED DESCRIPTION OF THE PREFERRED EMBODIMENTS

Referring now to FIGS. 2-7, various embodiments of a medical forced-air warming system 10 are shown. In a first illustrative embodiment, the medical forced-air warming system 10 comprises a table, bed or other support 12 for supporting a patient P above the ground. In the illustration being described, the medical forced-air warming system 10 comprises means, system and apparatus for delivering heated and decontaminated air to the patient P.

The medical forced-air warming system 10 comprises a decontamination system or means 20, a heater 24 and an air handler 14 that decontaminate and heat air which is supplied to a blanket 16 associated with the patient P. In the illustration being described, the blanket 16 comprises at least one or a plurality of apertures 16a to deliver the decontaminated and heated air to the patient P for warming the patient P. As illustrated, the heater 24 heats the air stream created by the air handler 14 and passes such air past the decontamination system or means 20, which comprises at least one or a plurality of irradiators or germicidal lamps 20a and 20b that irradiate and decontaminate the air before it is received in the blanket 16.

In the illustration being described, the blanket 16 comprises at least one or a plurality of apertures 16a that permit the decontaminated and heated air to warm or heat the blanket 16 and the patient P as the air passes therethrough, thereby providing warmed and decontaminated air to the patient P as illustrated in FIG. 2. The air handler 14 comprises a fan 22 and the heater 24, both of which are under the control of a controller (not shown). The heater 24 comprises at least one or and a plurality of heating coils 24a and 24b. Note in this example that the air handler 14 also houses the decontamination system or means 20 and the germicidal lamps 20a and 20b in the form of ultraviolet light sources.

In this regard, when the fan 22 is energized, it creates a negative pressure that pulls air into a housing 14a of the air handler 14 through an inlet (not shown) of the housing 14a. The fan 22 forces the air past the germicidal lamps 20a and 20b and into a conduit 18, which delivers the heated and irradiated air into the blanket 16. The air is forced by positive pressure through the blanket 16 and through the apertures 16a where the heated and decontaminated air is passed over the patient P as illustrated in FIG. 2, while some of the heated and decontaminated air is exhausted from the blanket 16 into the surrounding environment as illustrated in FIG. 2.

As illustrated and described later, it may be desirable to provide more or less perforations, exhaust ports or apertures 16a in the blanket 16 in order to control the amount of pressure in the blanket 16. Also, it may be desirable to provide no exhaust ports in the upper surface (as viewed in FIG. 2) of the blanket 16 so that all of the heated and decontaminated air is passed to the patient P.

Although not shown, it is contemplated that a switch, air regulator or pressure valve or alternatively to provide no exhaust ports or apertures 16a that face towards the patient P, so all air is exhausted away from the patient P.

It is important to understand that the decontamination system or means 20 is in contact with the air as it passes by the heater 24 and through the air handler 14 and decontaminates the air before it is passed or directed to and through the blanket 16. The treated air is exhausted from the blanket 16 and enters the environment after passing through the blanket 16. It is important to note that the medical forced-air warming system 10 captures the ambient air in the room, heats it and irradiates it before it is delivered to the blanket 16 and when that air is exhausted into the local environment, it is substantially free of any contamination, airborne bacteria or germs.

In the illustration being described, the air handler 14 may include the system, apparatus or features of the air handler and irradiation devices shown in U.S. Pat. Nos. 9,433,693; 9,457,119; 9,764,054; 10,039,854; 10,532,122 and 10,549,007; as well as U.S. Patent Publication Nos. 2018/0133084; 2018/0133355; 2018/0264391 and 2019/0099050, all of which are assigned to the same assignee as the present application and are incorporated herein by reference and made a part hereof.

As best illustrated in FIG. 2, the medical forced-air warming system 10 provides means, apparatus and system for decontaminating air and providing clean and heated air into the blanket 16 and ultimately to the patient P and also into the room or environment where the patient P is located. Advantageously, the medical forced-air warming system 10 facilitates reducing and/or eliminating germicides, contaminants and unwanted bacteria from coming into contact with the patient P while simultaneously providing heated air to the patient P. This, in turn, decreases chances of patient infection.

It should be understood that the air handler 14 therefor comprises germicidal radiation means or sources, such as one or more germicidal lamps 20a and 20b, which in the embodiment being described are at least one or a plurality of ultraviolet radiation lamps. It should be appreciated that the germicidal lamps 20a and 20b and the air handler 14 may comprise not only the germicidal lamps 20a and 20b, but can have other features which are shown and described in the above-referenced patents or patent applications, all of which are incorporated herein by reference and made a part hereof. For example, the air handler 14 or the germicidal lamps 20a and 20b may comprise air baffles, airflow interrupters for interrupting and disrupting the airflow, lenses, blockers, reflective surfaces, such as mirrors, or other similar means or apparatus to optimize the irradiation dosage and/or exposure duration of the air stream and/or suspended microbes therein, which in turn facilitates decontaminating the air stream as it flows through the air handler 14. The medical forced-air warming system 10 comprises a conventional control, as mentioned earlier, the air handler 14 and germicidal lamps 20a and 20b all of which are available from Aerobiotix, Inc. located in Miamisburg, Ohio.

Thus, in the illustration described relative to FIG. 2, the air handler 14 houses and comprises the germicidal lamps 20a and 20b in communication with the air stream as it flows therethrough. The air handler 14 comprises exhaust means comprising the germicidal lamps 20a and 20b which irradiate the air prior to exhausting into the blanket 16. In the illustration of FIG. 2, note that the germicidal lamps 20a and 20b are housed in the air handler 14.

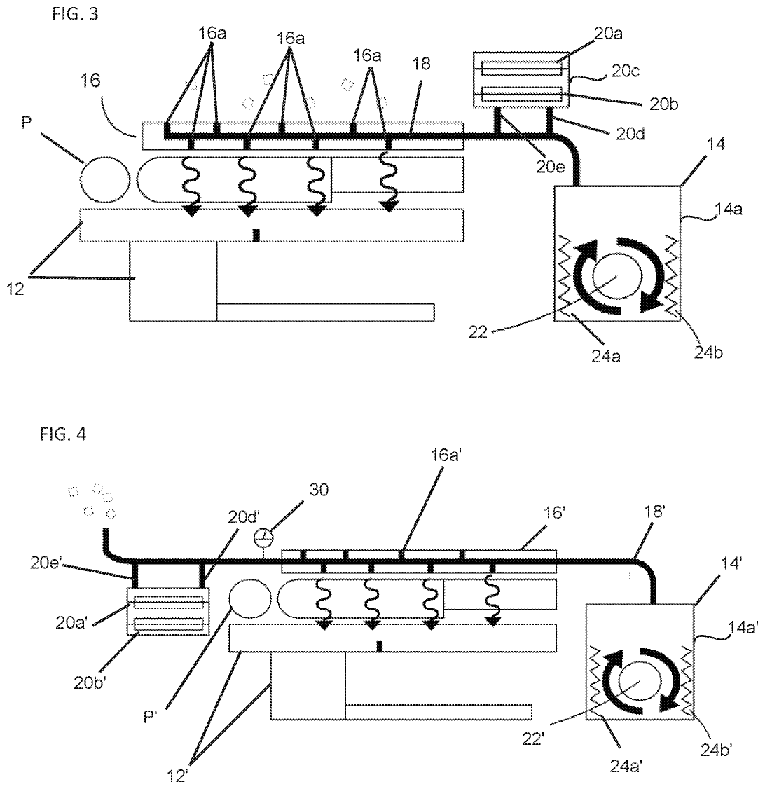

Referring now to the embodiment of FIG. 3, note that the decontamination system or means 20 is situated external to the air handler 14. In this regard, ambient air enters the air handler 14 which forces the air into the conduit 18 and to the decontamination system or means 20 whereupon the air stream is irradiated by the germicidal lamps 20a and 20b. In this embodiment, the decontamination system or means 20 comprises a housing 20c having an inlet 20d for receiving the forced air from the conduit 18. The housing 20c comprises at least one or a plurality of germicidal lamps 20a and 20b which are housed in the decontamination system or means 20. Thereafter, the air exits an outlet 20e and into the conduit 18 where it is delivered to the blanket 16. As with the embodiment described relative to FIG. 2, note that the air is heated and also decontaminated as it passes into the blanket 16 and onto and/or around the patient P. Air is also exhausted into the environment where the patient P is located as illustrated. Again, the blanket 16 comprises the apertures 16a that direct the heated and decontaminated airflow toward the patient P and also into the room.

Advantageously, these systems and embodiments shown and described relative to FIGS. 2 and 3 provide means, apparatus and a system for not only providing decontaminated and heated air to the patient P, but also for decontaminating the air before it enters into the room where the patient P is located.

FIGS. 4-7 illustrate other embodiments that will now be described. In these embodiments, the same parts are identified with the same part numbers, except that one or more prime mark(s) (“′”) have been added to these part numbers for the different embodiments.

Referring now to FIG. 4, still another embodiment is shown wherein the germicidal lamps 20a′ and 20b′ are situated downstream of the patient P′ whereupon they decontaminate the air before it is exhausted into the environment or room. In this regard, note that this embodiment comprises a pressure valve 30 for regulating the pressure and airflow through the passageway or conduit 18′ and blanket 16′ and before it enters the decontamination system or means 20′ and past the germicidal lamps 20a′ and 20b′. By contrast, note that the embodiments of FIGS. 2 and 3 decontaminate the air upstream of the patient P′ and before the air is delivered to the patient P′.

Yet another embodiment is shown in FIG. 5 which is similar to the embodiment shown in FIG. 2, except that some of the air in the conduit 18″ is returned to the air handler 14″ after it leaves the blanket 16″ as shown. In this embodiment, the pressure valve 30″ controls the airflow and pressure in the conduit 18″, which also permits control of the amount of clean air that is exhausted into the room and/or onto the patient P″. Thus, the exhaust from the blanket 16″ is under the control of and is controlled by the pressure valve 30″ and the medical forced-air warming system 10 returns the air to the air handler 14″ whereupon the air in the conduit 18″ is again irradiated and decontaminated. In this regard, the conduit 18″ comprises an inlet line 18a under positive pressure that delivers the heated and decontaminated air to the blanket 16″ and then a return line 18b under negative pressure that returns the air to the air handler 14″ as illustrated. By controlling the pressure valve 30″, the airflow and pressure through the conduit 18″ and the decontaminated and heated air delivered to the patient P″ and into the room can be controlled as well.

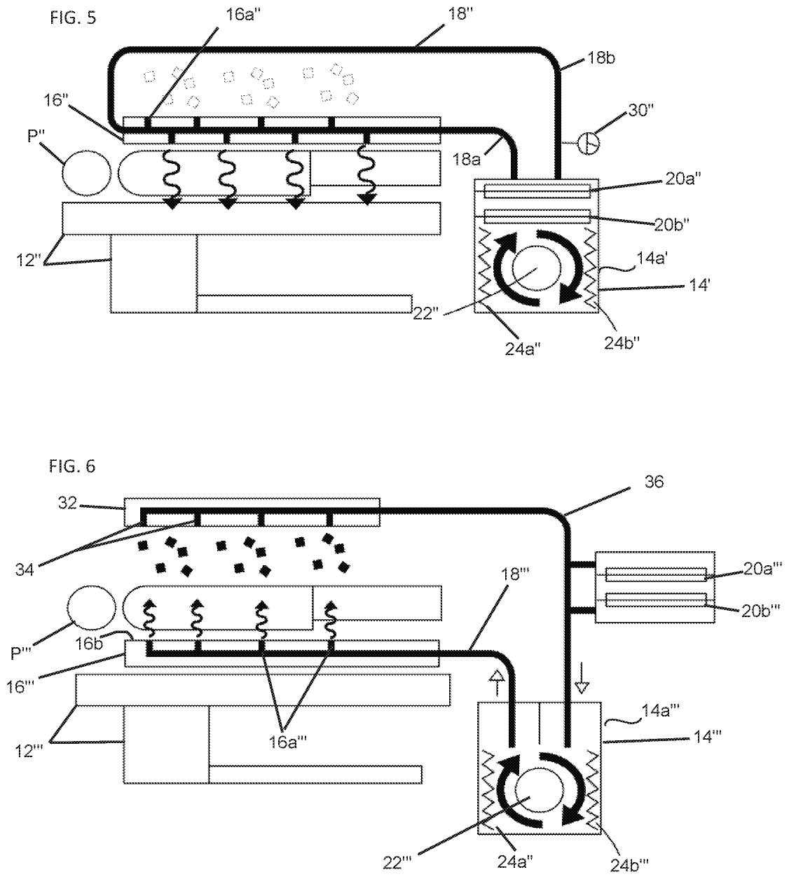

Referring now to FIG. 6, the blanket 16′″ comprises the exhaust apertures 16a′″ only on the surface 16b facing the patient P′″. The surface 16b is operatively associated with the patient P′″ so that heated and decontaminated air is delivered to the patient P′″ as illustrated. In this embodiment, a separate scavenger or collector 32 is provided that has a plurality of inlet apertures 34 for capturing any exhausted dirty or contaminated air, as well as any contaminated air in the environment around the patient P′″. A conduit 36 couples the scavenger or collector 32 to the air handler 14′″ which generates a negative pressure in the conduit 36 and at the plurality of inlet apertures 34 at the scavenger or collector 32. The negative pressure causes the exhausted dirty air and any contaminated air around the patient P′″ to be captured and then returned or delivered to the germicidal lamps 20a′″ and 20b′″ as illustrated.

Note that the embodiment of FIG. 6 is similar to the embodiment of FIG. 3 in that the germicidal lamps 20a′″ and 20b′″ are situated and housed outside of the air handler 14′″. In contrast, note that the embodiment of FIG. 5 is more similar to the embodiment of FIG. 2 wherein the germicidal lamps 20a″ and 20b″ are situated in the housing 14a″ of the air handler 14″ as shown.

Returning back to the description of the embodiment of FIG. 6, note that the air handler 14′″ generates positive pressure in the conduit 18′″ which forces the air into the blanket 16′″ and past the patient P′″ as illustrated. Substantially simultaneously, negative pressure is created by the air handler 14′″ in the conduit 36 which pulls the exhausted dirty air and any contaminated air surrounding the patient P into the scavenger or collector 32 whereupon it is delivered to the germicidal lamps 20a′″ and 20b′″ and ultimately returned to the air handler 14′″ and blanket 16′″ as illustrated. Similar to the embodiment of FIG. 4, the embodiment of FIG. 6 decontaminates and irradiates the air after heated and decontaminated air is delivered to the patient P′″. This embodiment is advantageous in that it facilitates capturing or scavenging dirty or contaminated air around the patient in the scavenger or collector 32 where it is then recycled and delivered to the patient P′″ via the conduit 18′″ after it is heated by the plurality of heating coils 24′″.

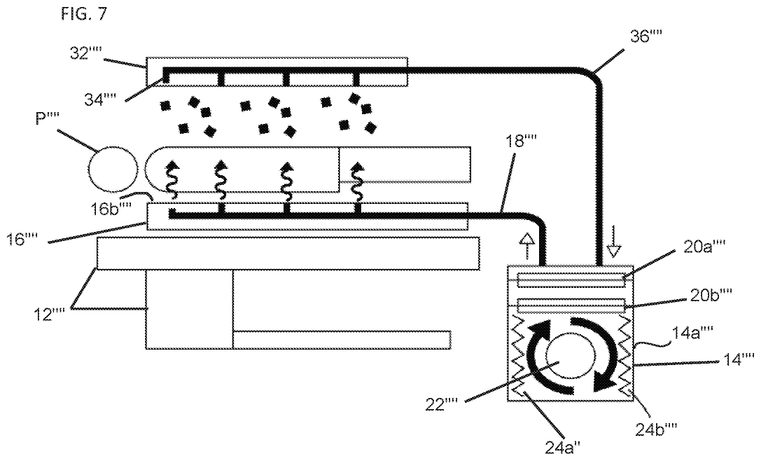

FIG. 7 (with a quadruple prime mark being used on similar part numbers to previous embodiments) illustrates still another embodiment similar to FIG. 6 except that the germicidal lamps 20a′″ and 20b′″ are situated in the air handler 14′″ similar to the embodiment of FIG. 2. Like the embodiment of FIG. 6, the medical forced-air warming system 10′″ captures the exhausted dirty air via negative pressure in the scavenger or collector 32′″ and any contaminated air around the patient P′″ and delivers it to the air handler 14′″ whereupon it is heated and then irradiated and decontaminated. Thereafter, the heated and decontaminated air is delivered through the conduit 18′″ to the blanket 16′″ where the air exits the apertures 16a′″ associated with surface 16b′″.

As with other embodiments, during operation, the fan 22″″ is energized and pulls air into the air handler 14″″ whereupon it is heated by the at least one or plurality of heating coils 24″″ and then irradiated and decontaminated by passing the air into the decontamination system or means 20″″ and past the germicidal lamps 20a″″ and 20b″″. The heated and decontaminated air is received and forced under positive pressure through the conduit 18″″ whereupon it is delivered to the blanket 16″″ and ultimately to the patient P″″ through the apertures 16a″″. Again, with this embodiment, the scavenger or collector 32″″ is utilized to capture exhausted dirty or contaminated air and any decontaminated air around the patient P″″ and delivers it via negative pressure to the air handler 14″″ as illustrated.

### ADDITIONAL CONSIDERATIONS

1. It should be understood that the air disinfection system shown and described in the various embodiments of FIGS. 2-7 provide means for irradiating and decontaminating the air utilizing at least one or a plurality of conventional ultraviolet light sources, such as C-band ultraviolet radiation sources, which have been shown to significantly reduce microbial levels in a moving air column. Applicant has found that by applying the principles of air stream irradiation to a forced-air patient warming blanket, Applicant has provided a significant system and means of improving patient health by reducing airborne contaminants, microbial levels, germs and the like while substantially simultaneously providing a warming system for warming the patient. It is believed that this not only facilitates patient comfort, but reduces the risk of infection to the patient by removing or substantially reducing the airborne contaminants that the patient is exposed to.

2. As mentioned earlier relative to the patents and patent applications cited earlier herein, Applicant's conventional air handlers 14 and heaters 24 can be used with the blanket 16 to achieve the advantages and objects described herein. They are available from Aerobiotix, Inc. located in Miamisburg, Ohio. Although not shown, such air handlers 14 and systems comprise the features shown and described in the aforementioned patents and patent applications, such as a conventional control system, power supplies, irradiation sources, and the like. In the illustrations being described, the heating coils 24 can be conventional heating coils which are adapted, mounted and housed in the housing 14a as illustrated in the Figures. They are also available from Aerobiotix, Inc. located in Miamisburg, Ohio

3. Although not shown, the heating coils 24 may also be provided in a separate housing, but it is preferable that they be housed in the air handler 14 as illustrated.

4. Note that in the embodiments of FIGS. 3 and 6, the germicidal lamps 20a and 20b are situated in a housing that is separate from the housing 14a. This is advantageous in some applications because it permits selective timing and control of the decontamination of the air. For example, FIGS. 3 and 6 illustrate that the germicidal lamps 20a and 20b can be situated downstream of the patient P and with the negative air pressure in the conduit 36, the exhausted dirty air and any contaminated air in the environment around the patient P can be captured by the scavenger or collector 32, treated and then delivered to the air handler 14 whereupon it is heated and then delivered to the patient P. In other embodiments, the germicidal lamps 20a and 20b are situated upstream of the patient P and treat the air prior to it being delivered to the patient P. This may be particularly useful when there is no scavenger or collector 32 or when all air passes through the blanket 16 and is exhausted into the room. In contrast, and as explained earlier relative to the embodiment of FIG. 4, the germicidal lamps 20a and 20b could be situated downstream of the patient P and treat the air before it is exhausted into the environment or room.

5. In the illustration being described, it is contemplated that the blanket 16 could be of any predetermined shape or size but preferably covers the entire patient P, and may even be large enough to completely wrap and surround the patient P. Likewise, the apertures 16a in the blanket 16 that permit the exhaust of the air towards the patient P and into the room may be selectively provided depending on how much air is being delivered to the patient P or is desired to be delivered to the patient P. In this regard, the one or more pressure valves 30 may be provided to control the airflow through the conduits 18 and 36 and in the medical forced-air warming system 10.

6. The germicidal lamps 20a and 20b are C-band ultraviolet lamps in the illustration being described and while the illustration shows a plurality of two or more germicidal lamps 20a and 20b, it should be appreciated that a single lamp 20a or 20b may be used or other means for treating the air stream. In this regard, chemical decontaminating filters, filter means or treatment means may be provided in addition to or in lieu of the germicidal lamps 20a and 20b. It is important that as much of the air that reaches the patient P be decontaminated so it may be necessary to provide more irradiation sources or other means for decontaminating the air before it reaches the patient P. Decontaminant components may also include: mechanical filtration or means, an irradiations system or means, a chemical decontamination means or an electrostatic device or means.

7. Although the various embodiments illustrate the patient P being situated on the table 12 or on the blanket 16 which is situated on the table 12, it should be understood that the medical forced-air warming system 10 may be utilized with a bed, chair or other device for supporting the patient P. Alternatively, the patient P may be in a non-prone position, such as a sitting or standing position, yet wrapped with the blanket 16. It is most preferred, however, that the blanket 16 be used particularly in pre-operative and post-operative procedures in order to reduce the chances of infection as a result of airborne contaminants or germs.

8. Although not shown, the blanket 16 may not be used and instead, the bed or other support 12 may be adapted and coupled to the medical forced-air warming system 10, so long as it is configured to receive and pass the warmed and decontaminated air.

9. Advantageously, the medical forced-air warming system 10 is particularly adapted for the medical environment, although it could be used outside of a medical environment, such as at home private use.

10. In some embodiments, the air stream is in two-way communication with positive and negative pressure between the air handler 14 and the blanket 16 and the communication comprises a return to the air handler 14, which in turn comprises the germicidal lamps 20a and 20b which irradiate the air stream returning from the blanket 16. Thus, the air stream is in two-way communication between the air handler 14 and the blanket 16 in some embodiments, and the air handler 14 handles outgoing and incoming air in a substantially simultaneous manner. This enables the air handler 14 to irradiate both incoming and outgoing air in order to substantially simultaneously decontaminate the air.

11. In still other embodiments, the germicidal lamps 20a and 20b are in communication with the air stream and comprise at least one or a plurality of irradiation sources in cooperation with air baffles, lenses, blockers, reflective surfaces, airflow interrupters or similar means or apparatus to optimize irradiation dosage and/or exposure duration of the air stream and/or suspended microbes therein. Applicant has found that by providing baffles and airflow interrupters, there is an increase in the irradiation dosage and exposure time of the air stream relative to the radiation sources which improves the decontamination and irradiation of the air stream. For example, the features of the devices shown in U.S. Patent Nos. U.S. Pat. Nos. 9,433,693; 9,457,119; 9,764,054; 10,039,854; 10,532,122 and 10,549,007; as well as U.S. Patent Publication Nos. 2018/0133084; 2018/0133355; 2018/0264391 and 2019/0099050, all of which are assigned to the same assignee as the present application and are incorporated herein by reference and made a part hereof.

12. Note that in another embodiment, a substantially tubular connection exists between the air handler 14 and the blanket 16 and the germicidal lamps 20a and 20b that are mounted within or in communication with the tubular connection. As shown, it should be appreciated that the germicidal lamps 20a and 20b may be separately housed from the air handler 14 if desired.

13. As mentioned earlier, one or more pressure valves or airflow control devices 30 may be provided in communication with the germicidal lamps 20a and 20b or with the decontamination heater and air handler 14. In this regard, the medical forced-air warming system 10 may comprise the exhaust means or apparatus to allow the air stream to exit the blanket 16, with the exhaust means comprising a pressure valve 30 in communication with the germicidal lamps 20a and 20b which irradiate the air prior to exiting the medical forced-air warming system 10. In some embodiments as shown, the air may be irradiated after the patient P has been exposed to it as well. It should be appreciated that the pressure valve 30, which may or may not be in communication with the germicidal lamps 20a and 20b that irradiate the air, may be adjusted to allow for a level of air return versus release from the blanket 16. The pressure valve 30 may also be adjusted to increase or decrease the amount of airflow and pressure in the conduits or passageways 18 or 36 in the medical forced-air warming system 10.

14. In still another embodiment, note that the blanket 16 comprises the apertures 16a under positive air pressure while the scavenger or collector 32 scavenges air through air apertures 34 under negative air pressure to capture the air and return it to the decontamination heater and air handler 14. The air can be treated external of the air handler 14 or as with the embodiments shown in FIG. 2, for example, the air can be treated inside of the decontamination heater and air handler 14. As mentioned earlier, the blanket 16 comprises the apertures 16a under positive air pressure while the scavenger or collector 32 comprises the plurality of apertures 34 under negative air pressure which captures contaminated air or exhausted air from the blanket 16 and contaminated air surrounding the patient P. The contaminated air may be treated externally by the germicidal lamps 20a and 20b, which may be external to the air handler 14 or housed in the air handler 14 as described in the various embodiments.

15. In the illustration, note that the scavenger or collector 32 is situated in proximity to the patient P being warmed and may comprise or house the at least one or a plurality of germicidal lamps 20a and 20b which irradiate the scavenged and collected air. Thus, it is contemplated that the germicidal lamps 20a and 20b may be housed in the scavenger or collector 32 rather than housed separately or housed in the decontamination heater and air handler 14. This is particularly useful for adapting the medical forced-air warming system 10 to conventional environments, which facilitates preventing the release of contaminated air into the environment.

16. While the embodiment is shown as delivering the decontaminated and heated air to the blanket 16, other devices or apparel may be used in association with this medical forced-air warming system 10 in order to reduce patient infection resulting from airborne contaminants. In this regard, the medical forced-air warming system 10 may be used with a mask, patient clothing apparel, patient tent or the like.

17. Advantageously, the medical forced-air warming system 10 provides means, an apparatus and a system for decontaminating and heating air that is delivered to the blanket 16 and the patient P. It should be understood, however, that it is not necessary for the air to be substantially simultaneously heated and decontaminated, these can occur in different steps. In fact, the heating and decontamination can occur in two separate units or stages within the system. The main goal is to prevent release of contaminated air into the room environment, not necessarily delivery of clean air to the patient which is an added benefit of the system. Because the blanket is non-perforated, the patient is not directly exposed to the air supply. It is only when air is exhausted at the control unit into the environment that decontamination is needed. This exhausting occurs when there is too much air in the system, such as flexing or compressing the blanket (like squeezing a balloon).

18. A key feature of this system is maintain flexibility and inflation of the blanket in any conformation or under compression, without leakage of air around the patient. In contrast, the prior art uses perforations in the blanket to achieve this function. This invention moves that exhaust air over the control unit where it can be filtered or decontaminated.

Advantageously, the embodiments shown and described herein could be used alone or together and/or in combination with one or more of the features covered by one or more of the claims set forth herein, including but not limited to one or more of the features or steps mentioned in the Summary of the Invention and the claims.

While the system, apparatus and method herein described constitute preferred embodiments of this invention, it is to be understood that the invention is not limited to this precise system, apparatus and method, and that changes may be made therein without departing from the scope of the invention which is defined in the appended claims.

What is claimed is:

1. A medical warming system comprising:
an air handler;
a heater for heating air generated or received by said air handler;
a blanket adapted to be placed on or near a patient, said blanket having at least one air channel for receiving an air stream generated by said air handler, said blanket having at least one perforation, exhaust or aperture for permitting at least some of said air stream received by said blanket to exit said blanket; and
a germicidal radiation source for decontaminating said air stream generated or received by said air handler.

2. The medical warming system as recited in claim 1 wherein said germicidal radiation source is located upstream of said patient so that said air stream generated by said air handler is decontaminated before said air stream reaches said blanket.

3. The medical warming system as recited in claim 2 wherein said germicidal radiation source is located downstream of said patient so that said air stream is decontaminated by said germicidal radiation source after said air stream passes through said blanket.

4. The medical warming system as recited in claim 1 wherein said heater and said germicidal radiation source are situated in said air handler.

5. The medical warming system as recited in claim 1 wherein said medical warming system further comprises a conduit or air tube for receiving said air stream generated by said air handler and for delivering said air stream to said at least one air channel of said blanket, said germicidal radiation source being in fluid communication with said conduit or air tube and situated between said air handler and said blanket and irradiating at least some of said air stream generated by said air handler before it enters in said blanket.

6. The medical warming system as recited in claim 1 wherein said medical warming system further comprises a conduit or air tube for receiving said air stream generated by said air handler and for delivering said air stream to said at least one air channel of said blanket and, thereafter, to an environment, said germicidal radiation source being in fluid communication with said conduit or air tube and adapted to receive and irradiate at least a portion of said air stream after said air stream exits said blanket and before said air stream is introduced into said environment.

7. The medical warming system as recited in claim 6 wherein said conduit or air tube comprises at least one adjustable valve for controlling pressure or airflow through said blanket.

8. The medical warming system as recited in claim 1 wherein said germicidal radiation source comprises an irradiation optimizer for optimizing irradiation dosage and/or exposure duration of said air stream to irradiate microbes suspended therein.

9. The medical warming system as recited in claim 8 wherein said irradiation optimizer comprises at least one of air baffles, airflow interrupters, lenses, blockers or reflective surfaces.

10. The medical warming system as recited in claim 1 wherein said germicidal radiation source irradiates said air stream such that substantially decontaminated air is introduced into an environment around said patient.

11. The medical warming system as recited in claim 1 wherein said germicidal radiation source comprises at least one ultraviolet lamp.

12. The medical warming system as recited in claim 1 wherein said germicidal radiation source comprises a plurality of ultraviolet lamps.

13. The medical warming system as recited in claim 2 wherein said germicidal radiation source is situated in said air handler.

14. A medical warming system comprising:
an air handler;
a heater for heating an air stream generated or received by said air handler;
a blanket adapted to be placed on or near a patient, said blanket having at least one air channel for receiving said air stream generated by said air handler, said blanket having at least one exhaust for permitting at least some of said air stream received by said blanket to exit said blanket;
a germicidal radiation source for decontaminating said air stream generated or received by said air handler;
said exhaust comprising said germicidal radiation source which irradiates said air stream after said air stream passes through said blanket and prior to said air stream being exhausted from said medical warming system.

15. The medical warming system as recited in claim 14 wherein said germicidal radiation source is located downstream of said patient so that said air stream is decontaminated by said germicidal radiation source after said air stream passes through said blanket.

16. The medical warming system as recited in claim 15 wherein said medical warming system further comprises a conduit or air tube for receiving said air stream generated by said air handler and for delivering said air stream to said at least one air channel of said blanket and, thereafter to an environment, said germicidal radiation source being in fluid communication with said conduit or air tube and adapted to receive and irradiate at least a portion of said air stream after said air stream exits said blanket and before said air stream is exhausted from said medical warming system.

17. The medical warming system as recited in claim 16 wherein said conduit or air tube comprises at least one adjustable valve for controlling pressure or airflow through said blanket.

18. The medical warming system as recited in claim 14 wherein said germicidal radiation source comprises an irradiation optimizer for optimizing irradiation dosage and/or exposure duration of said air stream to irradiate microbes suspended therein.

19. The medical warming system as recited in claim 18 wherein said irradiation optimizer comprises at least one of air baffles, airflow interrupters, lenses, blockers or reflective surfaces.

20. The medical warming system as recited in claim 14 wherein said germicidal radiation source comprises at least one ultraviolet lamp.

21. The medical warming system as recited in claim 14 wherein said germicidal radiation source comprises a plurality of ultraviolet lamps.

22. A medical warming system comprising:
an air handler;
a heater for heating an air stream generated or received by said air handler;
a warming blanket adapted to be placed on or near a patient, said warming blanket having at least one air channel for receiving said air stream generated by said air handler, said warming blanket having at least one perforation, exhaust or aperture for permitting at least some of said air stream received by said warming blanket to exit said warming blanket;
a germicidal radiation source for decontaminating said air stream generated or received by said air handler;
a communication conduit comprising a delivery conduit for coupling an outlet of said air handler to an inlet of said at least one air channel of said warming blanket and a return conduit for coupling an outlet of said at least one air channel of said warming blanket to an inlet of said air handler, said germicidal radiation source being situated in at least one of said delivery conduit or said return conduit.

23. The medical warming system as recited in claim 22 wherein said germicidal radiation source is located in said return conduit so that said air stream is decontaminated by said germicidal radiation source after said air stream passes through said warming blanket and before said air stream passes into said inlet of said air handler.

24. The medical warming system as recited in claim 23 wherein said medical warming system further comprises a conduit or air tube for receiving said air stream generated by said air handler and for delivering said air stream to said at least one air channel of said warming blanket and, thereafter to an environment, said germicidal radiation source being in fluid communication with said conduit or air tube and adapted to receive and irradiate at least a portion of said air stream after said air stream exits said warming blanket and before said air stream is exhausted from said medical warming system.

25. The medical warming system as recited in claim 24 wherein said medical warming system comprises at least one adjustable valve for controlling pressure or airflow through said warming blanket.

26. The medical warming system as recited in claim 25 wherein said at least one adjustable valve is situated in said return conduit.

27. The medical warming system as recited in claim 22 wherein said germicidal radiation source comprises an irradiation optimizer for optimizing irradiation dosage and/or exposure duration of said air stream to irradiate microbes suspended therein.

28. The medical warming system as recited in claim 27 wherein said irradiation optimizer comprises at least one of air baffles, airflow interrupters, lenses, blockers or reflective surfaces.

29. The medical warming system as recited in claim 22 wherein said germicidal radiation source comprises at least one ultraviolet lamp.

30. The medical warming system as recited in claim 22 wherein said germicidal radiation source comprises a plurality of ultraviolet lamps.

31. A medical warming system comprising:
an air handler;
a warming blanket adapted to be placed on or near a patient, said warming blanket having at least one air channel for receiving an air stream generated by said air handler, said warming blanket having at least one perforation, exhaust or aperture for permitting at least some of said air stream received by said warming blanket to exit said warming blanket; and
a germicidal radiation source for decontaminating said air stream generated or received by said air handler;
said air handler generating said air stream that is in two way communication between said air handler and said warming blanket, so that said air handler generates an outgoing air stream and substantially simultaneously receives an incoming air stream from said warming blanket;
said germicidal radiation source irradiating both said incoming air stream into said air handler and said outgoing air stream from said air handler substantially simultaneously.

32. The medical warming system as recited in claim 31 wherein said medical warming system comprises a communication conduit comprising a delivery conduit for coupling an outlet of said air handler to an inlet of said at least one air channel of said warming blanket and a return conduit for coupling an outlet of said at least one channel of said warming blanket to an inlet of said air handler, said germicidal radiation source being situated in at least one of said delivery conduit or said return conduit.

33. The medical warming system as recited in claim 31 wherein said germicidal radiation source is located in said air handler so both air entering and exiting said air handler are subject to irradiation by said germicidal radiation source.

34. The medical warming system as recited in claim 31 wherein said medical warming system further comprises a heater situated in said air handler for heating said air stream generated by said air handler.

35. The medical warming system as recited in claim 32 wherein said medical warming system comprises at least one adjustable valve for controlling pressure or airflow through said warming blanket.

36. The medical warming system as recited in claim 35 wherein said at least one adjustable valve is situated in said return conduit.

37. The medical warming system as recited in claim 31 wherein said germicidal radiation source comprises an irradiation optimizer for optimizing irradiation dosage and/or exposure duration of said air stream to irradiate microbes suspended therein.

38. The medical warming system as recited in claim 37 wherein said irradiation optimizer comprises at least one of air baffles, airflow interrupters, lenses, blockers or reflective surfaces.

39. The medical warming system as recited in claim 31 wherein said germicidal radiation source comprises at least one ultraviolet lamp.

40. The medical warming system as recited in claim 31 wherein said germicidal radiation source comprises a plurality of ultraviolet lamps.

41. A medical warming system comprising:
an air handler;
a warming blanket adapted to be placed on or near a patient, said warming blanket having at least one air channel for receiving an air stream generated by said air handler, said warming blanket having at least one perforation, exhaust or aperture for permitting at least some of said air stream received by said warming blanket to exit by warming blanket via positive pressure;
a germicidal radiation source for decontaminating by air stream generated or received by said air handler; and
a scavenger or collector in fluid communication with said air handler, said scavenger or collector being situated in proximity to said patient and capturing said air stream under negative pressure generated by said air handler and returning said air stream to said air handler, said scavenger or collector comprising said germicidal radiation source which irradiates said air stream collected by said scavenger or collector.

42. The medical warming system as recited in claim 41 wherein said germicidal radiation source is located in said air handler and irradiates said air stream received in said air handler so that said air stream is decontaminated by said germicidal radiation source after said air stream passes through said warming blanket and before said air stream passes out of said air handler.

43. The medical warming system as recited in claim 41 wherein said medical warming system comprises a collector conduit coupling said scavenger or collector to said air handler, said germicidal radiation source being in communication with said collector conduit so that said air stream is decontaminated by said germicidal radiation source after said air stream passes through said warming blanket and before said air stream passes into said air handler.

44. The medical warming system as recited in claim 41 wherein said scavenger or collector comprises a plurality of inlets for capturing said air stream and for delivering said air stream to said germicidal radiation source where said air stream is irradiated.

45. The medical warming system as recited in claim 41 wherein a system heater is located in said air handler for heating said air stream.

46. The medical warming system as recited in claim 41 wherein a system heater is located in said warming blanket.

47. The medical warming system as recited in claim 43 wherein said medical warming system comprises a communication conduit comprising a delivery conduit for coupling an outlet of said air handler to an inlet of said at least one air channel of said warming blanket and a return conduit for coupling to an outlet of said scavenger or collector to an inlet of at least one of an inlet of said air handler or an inlet of said germicidal radiation source.

48. The medical warming system as recited in claim 41 wherein said scavenger or collector comprises scavenging means for scavenging and collecting air discharged from said warming blanket, said scavenging means comprising said germicidal radiation source and a perforated generally planar collector.

49. The medical warming system as recited in claim 43 wherein said germicidal radiation source is located in said collector conduit so that said air stream is decontaminated by said germicidal radiation source after said air stream passes through said warming blanket and before said air stream passes into an inlet of said air handler.

50. The medical warming system as recited in claim 42 wherein said medical warming system further comprises a conduit or air tube for receiving said air stream generated by said air handler and for delivering said air stream to said at least one air channel of said warming blanket and, thereafter, to said germicidal radiation source which is in fluid communication with said conduit or air tube and adapted to receive and irradiate at least a portion of said air stream after said air stream exits said warming blanket.

51. The medical warming system as recited in claim 41 wherein said germicidal radiation source comprises an irradiation optimizer for optimizing irradiation dosage and/or exposure duration of said air stream to irradiate microbes suspended therein.

52. The medical warming system as recited in claim 51 wherein said irradiation optimizer comprises at least one of air baffles, airflow interrupters, lenses, blockers or reflective surfaces.

53. The medical warming system as recited in claim 41 wherein said germicidal radiation source comprises at least one ultraviolet lamp.

54. The medical warming system as recited in claim 41 wherein said germicidal radiation source comprises a plurality of ultraviolet lamps.

55. A medical forced air warming system comprising:
an air handler comprising a warming means;
a warming blanket with air channels;
an air stream between and inclusive of said air handler and said blanket; and
a germicidal radiation source;
said radiation source in communication with said airstream.

56. A medical forced air warming system comprising:
an air handler comprising a warming means;
a warming blanket with air channels;
an air stream between and inclusive of said air handler and said blanket; and
an exhaust means to allow said air stream to exit said blanket;
said exhaust means comprising germicidal radiation source which irradiates said air prior to exhausting from said system.

57. A medical forced air warming system comprising:
an air handler comprising a warming means;
a warming blanket with air channels; and
an air stream in two way communication between said air handler and said blanket;
said communication comprising a return to said air handler;
said air handler further comprising a germicidal irradiation means to irradiate said air stream retuning from said blanket.

58. A medical forced air warming system comprising:
an air handler comprising a warming means;
a warming blanket with air channels; and
an air stream in two way communication between said air handler and said blanket;
said air handler handling outgoing and incoming air in communication with said blanket;
said air handler further comprising a germicidal irradiation means irradiating said incoming and said outgoing air in a substantially simultaneous manner.

59. A medical forced air warming system comprising:
an air handler comprising a warming means;
a warming blanket with air channels;
an air stream between and inclusive of said air handler and said blanket; and
a germicidal radiation means;
said radiation means in communication with said airstream;
said radiation means comprising a radiation source in cooperation with air baffles, lenses, blockers, reflective surfaces, or similar means to optimize irradiation dosage and/or exposure duration of said air stream and/or suspended microbes therein.

60. A medical forced air warming system comprising:
an air handler comprising a warming means;
a warming blanket comprising air channels;
a substantially tubular connection between said air handler and said blanket; and
a germicidal radiation source;
said radiation source mounted within, or in communication with, said tubular connection.

61. A medical forced air warming system comprising:
an air handler comprising a warming means;
a warming blanket comprising air channels;
an air stream between and inclusive of said air handler and said blanket; and
an exhaust means to allow said air stream to exit said blanket;
said exhaust means comprising pressure valve and in communication with a germicidal radiation source which irradiates said air prior to exhausting from said system.

62. A medical forced air warming system comprising:
an air handler comprising a warming means;
a warming blanket comprising air channels;
an air stream between and inclusive of said air handler and said blanket; and
an exhaust means to allow said air stream to exit said blanket;
said exhaust means comprising pressure valve and in communication with a germicidal radiation source which irradiates said air;
said pressure valve adjustable to allow for level of air return versus release from said blanket.

63. A medical forced air warming system comprising:
an air handler comprising a warming means;
a warming blanket comprising air apertures under positive air pressure; and
a scavenging means comprising air apertures under negative air pressure;
said scavenging means in proximity to subject being warmed and comprising a germicidal radiation source which irradiates said scavenged air.
